(19) United States
(12) Patent Application Publication
(10) Pub. No.: US 2018/0055113 A1
(43) Pub. Date: Mar. 1, 2018

(54) PROTECTIVE GLOVES WITH PROTRUDING PORTION

(71) Applicant: Roger Barranco, Pompano Beach, FL (US)

(72) Inventor: Roger Barranco, Pompano Beach, FL (US)

(21) Appl. No.: 15/254,699

(22) Filed: Sep. 1, 2016

(51) Int. Cl. A41D 19/00; A61B 42/10; A61B 42/50; A47G 25/90

(52) U.S. Cl. CPC A41D 19/0075; A41D 19/0044; A47G 25/904; A61B 42/10; A61B 42/50; A41D 19/0082

(57) ABSTRACT

A pair of protective gloves is disclosed, wherein each glove comprises: (a) a hollow-formed, seamless and flexible body element composed of a polymer, the flexible body element including a closed distal end for containing the wearer's hand, an open proximal end, and a cuff portion, (b) a bulging portion of the flexible body element located on the cuff portion, said bulging portion extending around the cuff to form a ring around the cuff, and said bulging portion projecting outwards from said body element, (c) wherein said bulging portion comprising loose material that may be pinched between the wearer's fingers, and (d) wherein said bulging portion being positioned so that when said glove is properly positioned on the wearer's hand, said bulging portion may be pulled in a substantially distal direction, thereby removing the glove from the wearer's hand without the exterior surface of the glove touching the wearer's hand.

### CROSS-REFERENCE TO RELATED APPLICATIONS

Not Applicable.

### STATEMENT REGARDING FEDERALLY SPONSORED RESEARCH OR DEVELOPMENT

Not Applicable.

### INCORPORATION BY REFERENCE OF MATERIAL SUBMITTED ON A COMPACT DISC

Not Applicable.

### TECHNICAL FIELD

The technical field relates generally to protective gloves and, more specifically, to occupational gloves for protecting the wearer from contamination.

### BACKGROUND

Protective gloves have been used extensively in numerous fields, such as the medical field where they are used by doctors, nurses, hospital employees and laboratory technicians. Protective gloves in the medical field are used for surgical procedures, medical examinations, treating patients having contagious diseases, blood drawing, testing and transfusions, and numerous other surgical, examination, medical and laboratory procedures. Due to the increased concern regarding diseases which may be transferred by contact from person to person, gloves of this type are now being used by dentists, police officers, firefighting personnel, beauticians and even highway toll booth attendants.

Conventional protective gloves, however, sometimes allow an opportunity for foreign materials to gain entrance to the glove's interior thus compromising the amount of protection the glove can provide. A loose glove cuff may tend to fall down the wearer's wrist, reducing protection and presenting an opportunity to be inadvertently stripped from the user's hand during active use. Conversely, an overly tight cuff, such as can be produced by a conventional rubber glove or rubber band inserted in the cuff roll of a plastic glove, may not be desirable either. With its high elastic memory, such a glove could not only cause difficulty in donning but provide increasingly undesirable effects to the user due to possible blood circulation restriction. A glove when worn must be such that it will not impair circulation in the wrist area because such impairment may affect the touch or tactile sense of the hand and the sensitivity of the hand to heat and cold and could possibly provoke hand and finger numbness when worn for a prolonged amount of time such as in a surgical procedure. Further, conventional methods for removing medical gloves often involves steps that may contaminate the wearer's bare hands with material that may exist on the exterior surface of the glove. This defeats the purpose of the protective glove.

Therefore, a need exists for improvements over the prior art, and, more particularly, for safer and more efficient methods and devices for donning and removing protective gloves.

### SUMMARY

An improved pair of protective gloves is provided. This Summary is provided to introduce a selection of disclosed concepts in a simplified form that are further described below in the Detailed Description including the drawings provided. This Summary is not intended to identify key features or essential features of the claimed subject matter. Nor is this Summary intended to be used to limit the claimed subject matter's scope.

In one embodiment, a pair of protective gloves is disclosed, wherein each glove comprises: (a) a hollow-formed, seamless and flexible body element composed of a polymer, the flexible body element including a closed distal end for containing the wearers hand, an open proximal end, and a cuff portion at said proximal end, wherein the flexible body element is configured to conform tightly to the wearer's hand, (b) a bulging portion of the flexible body element located on the cuff portion, said bulging portion extending around the cuff to form a ring around the cuff, and said bulging portion projecting outwards from said body element so as not to conform to the wearer's hand, (c) wherein said bulging portion comprising loose material that may be pinched between the wearer's fingers, so as to allow significant purchase on the glove, and (d) wherein said bulging portion being positioned so that when said glove is properly positioned on the wearers hand, said bulging portion may be pulled in a substantially distal direction, thereby removing the glove from the wearer's hand without the exterior surface of the glove touching the wearer's hand.

### BRIEF DESCRIPTION OF THE DRAWINGS

The accompanying drawings, which are incorporated in and constitute a part of this disclosure, illustrate various example embodiments. In the drawings:

FIG. 1 is an illustration of a perspective view of a first embodiment of the claimed protective gloves, according to an example embodiment;

FIG. 2A is an illustration of a perspective view of a second embodiment of the claimed protective gloves, according to an example embodiment;

FIG. 2B is an illustration of a perspective view of a third embodiment of the claimed protective gloves, according to an example embodiment;

FIG. 3 is an illustration of a perspective view of a fourth embodiment of the claimed protective gloves, according to an example embodiment;

FIG. 4 is an illustration showing how the first embodiment of the claimed protective gloves may be donned, according to an example embodiment;

FIG. 5 is an illustration showing how the first embodiment of the claimed protective gloves may be removed, according to an example embodiment;

FIGS. 6A through 6C are illustrations showing the process of donning a conventional medical glove.

FIGS. 7A through 7C are illustrations showing the process of removing a conventional medical glove.

FIG. 8A is an illustration of the beginning of the process of removing the claimed protective gloves, according to an example embodiment;

FIG. 8B is an illustration of the middle of the process of removing the claimed protective gloves, according to an example embodiment; and

FIG. 8C is an illustration of further in the process of removing the claimed protective gloves, according to an example embodiment.

### DETAILED DESCRIPTION

The following detailed description refers to the accompanying drawings. Wherever possible, the same reference numbers are used in the drawings and the following description to refer to the same or similar elements. While embodiments herein may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the claimed subject matter. Instead, the proper scope of the claimed subject matter is defined by the appended claims.

The claimed subject matter improves over the prior art by providing a more efficient, safe and precise way of donning and removing protective gloves. The claimed subject matter utilizes a bulging portion on the protective gloves to allow a wearer, or a companion, to quickly and easily don, or remove, a glove on, or from, the wearer, without risk of contamination. The claimed subject matter protects the wearer or a companion from contamination without introducing complex or costly procedures or equipment. The claimed subject matter is further easy to manufacture and the method for donning and removing the gloves is simple to understand for users.

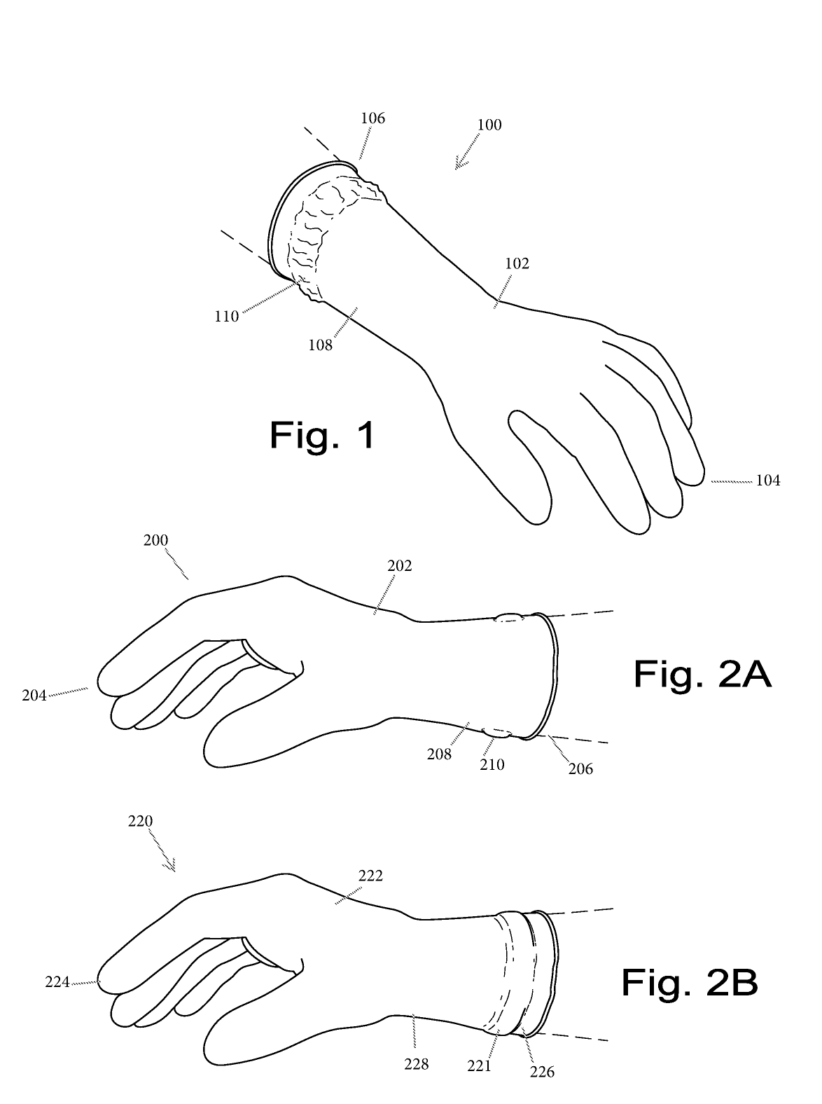

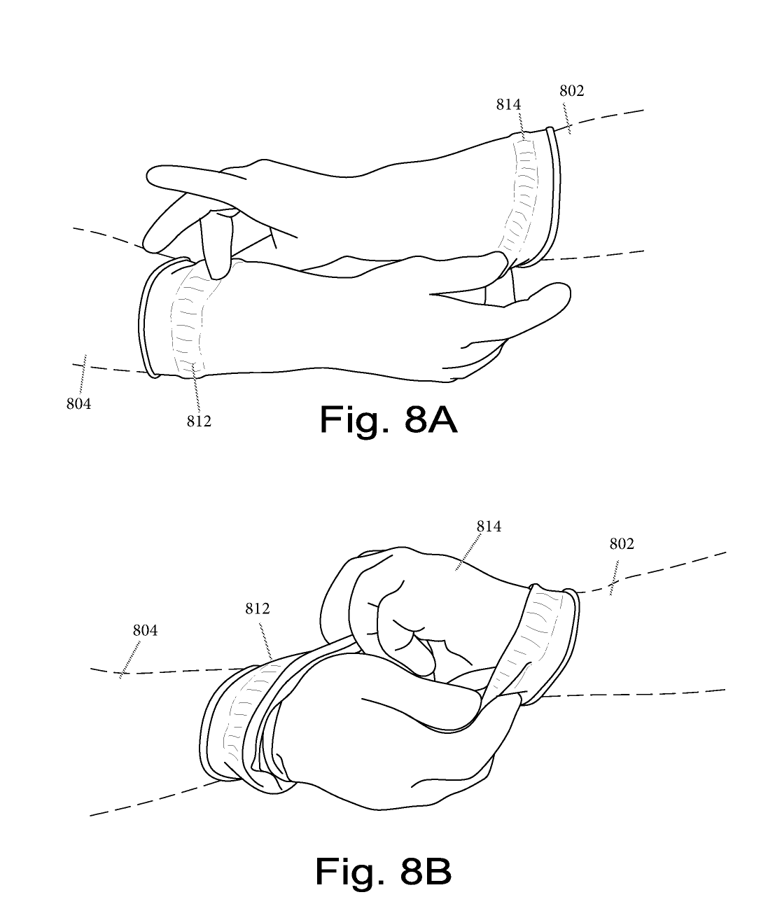

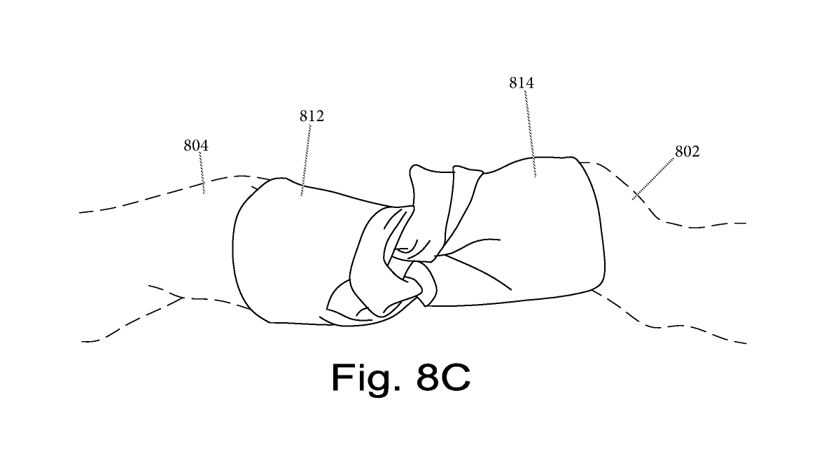

FIG. 1 is an illustration of a perspective view of a first embodiment 100 of the claimed protective gloves, according to an example embodiment. The first embodiment 100 includes a hollow-formed, seamless and flexible body element 102 composed of a polymer, the flexible body element including a closed distal end 104 for containing the wearer's hand, an open proximal end 106, and a cuff portion 108 at said proximal end, wherein the flexible body element is configured to conform tightly to the wearer's hand. The first embodiment 100 also includes a bulging portion 110 of the flexible body element located on the cuff portion, said bulging portion extending around the cuff to form a ring around the cuff, and said bulging portion projecting outwards from said body element so as not to conform to the wearer's hand. The bulging portion 110 comprises loose material that may be pinched between the wearer's fingers, so as to allow significant purchase on the glove. The bulging portion 110 is positioned so that when said glove is properly positioned on the wearer's hand, said bulging portion may be pulled in a substantially distal direction (see FIGS. 8A through 8C), thereby removing the glove from the wearer's hand without the exterior surface of the glove touching the wearer's hand. The bulging portion 110 may comprise irregularly or regularly shaped loose material that encircles the cuff portion.

FIG. 2A is an illustration of a perspective view of a second embodiment 200 of the claimed protective gloves, according to an example embodiment. Similar to the first embodiment 100, the second embodiment 200 also includes a hollow-formed, seamless and flexible body element 202 including a closed distal end 204 for containing the wearer's hand, an open proximal end 206, and a cuff portion 208 at said proximal end. The second embodiment 200 also includes a bulging portion 210 of the flexible body element located on the cuff portion, said bulging portion extending around the cuff, and said bulging portion projecting outwards from said body element so as not to conform to the wearer's hand. The bulging portion 210 comprises loose material that may be pinched between the wearer's fingers, so as to allow significant purchase on the glove. The bulging portion 210 may comprise two elongated tabs of loose material on opposite sides of the cuff portion.

FIG. 2B is an illustration of a perspective view of a third embodiment 220 of the claimed protective gloves, according to an example embodiment. Similar to the first embodiment 100, the third embodiment 220 also includes a hollow-formed, seamless and flexible body element 222 including a closed distal end 224 for containing the wearer's hand, an open proximal end 226, and a cuff portion 228 at said proximal end. The third embodiment 220 also includes a bulging portion 221 of the flexible body element located on the cuff portion, said bulging portion extending around the cuff to form a ring, and said bulging portion projecting outwards from said body element so as not to conform to the wearer's hand. The bulging portion 221 comprises loose material that may be pinched between the wearer's fingers, so as to allow significant purchase on the glove. The bulging portion 221 may comprise a continuous ring around the cuff portion, wherein the ring is elevated from the skin of the wearer's hand, as compared to the skintight or body-forming nature of the remainder of the glove.

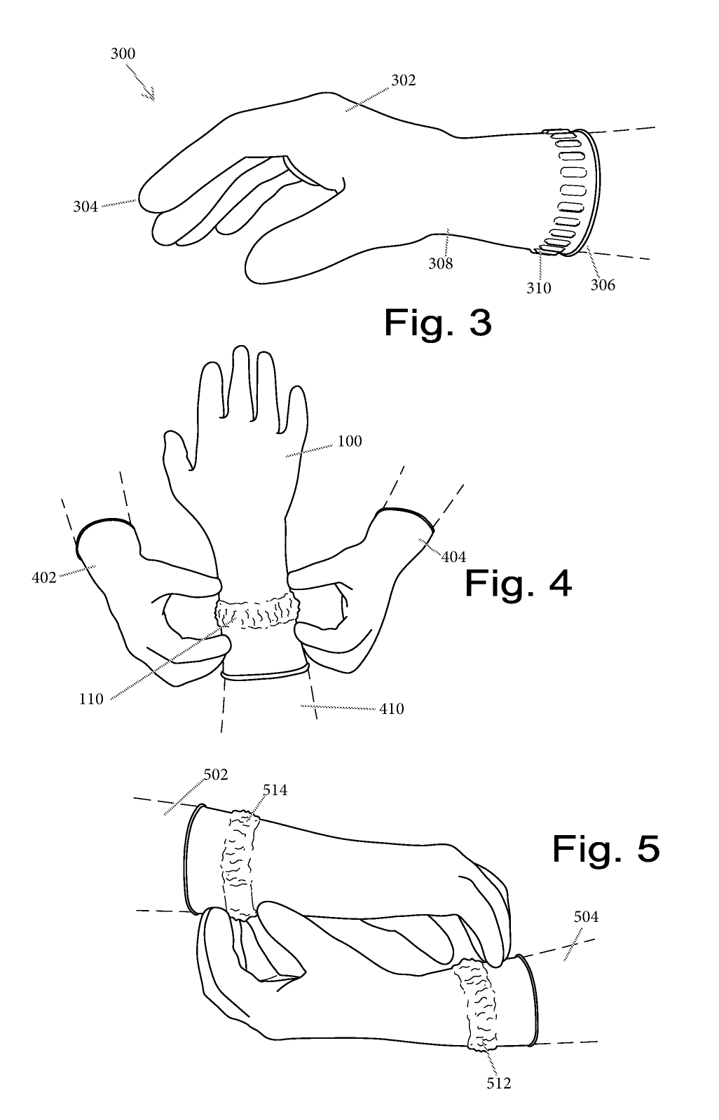

FIG. 3 is an illustration of a perspective view of a fourth embodiment 300 of the claimed protective gloves, according to an example embodiment. Similar to the first embodiment 100, the fourth embodiment 300 also includes a hollow-formed, seamless and flexible body element 302 including a closed distal end 304 for containing the wearer's hand, an open proximal end 306, and a cuff portion 308 at said proximal end. The fourth embodiment 300 also includes a bulging portion 310 of the flexible body element located on the cuff portion, said bulging portion extending around the cuff to form a ring, and said bulging portion projecting outwards from said body element so as not to conform to the wearer's hand. The bulging portion 310 comprises loose material that may be pinched between the wearer's fingers, so as to allow significant purchase on the glove. The bulging portion 310 may comprise a set of elliptical shaped protrusions that extend around the cuff, wherein the elliptical shaped protrusions are elevated from the skin of the wearer's hand.

FIG. 4 is an illustration showing how the first embodiment 100 of the claimed protective gloves may be donned, according to an example embodiment. FIG. 4 shows how a companion may use his/her hands 402, 404 to aid the wearer 410 in donning the glove. FIG. 4 shows how the bulging portion 110 may be pinched between the companion's fingers, so as to allow significant purchase on the glove. The bulging portion 110 may be pulled in a substantially proximal direction, thereby donning the glove onto the wearer's hand without the exterior surface of the glove touching the wearer's or the companion's bare hands.

FIG. 5 is an illustration showing how the first embodiment 100 of the claimed protective gloves may be removed, according to an example embodiment. FIG. 5 shows how the wearer may use a first hand 502 to grab the bulging portion 512 on the second hand 504, so as to pinch the bulging portion between his/her fingers, and allow significant purchase on the glove on the second hand 504. FIG. 5 also shows how the wearer may use his/her second hand 504 to grab the bulging portion 514 on the first hand 502, so as to pinch the bulging portion between his/her fingers, and allow significant purchase on the glove on the first hand 502. The glove removal process is explained in more detail with reference to FIGS. 8A through 8C below.

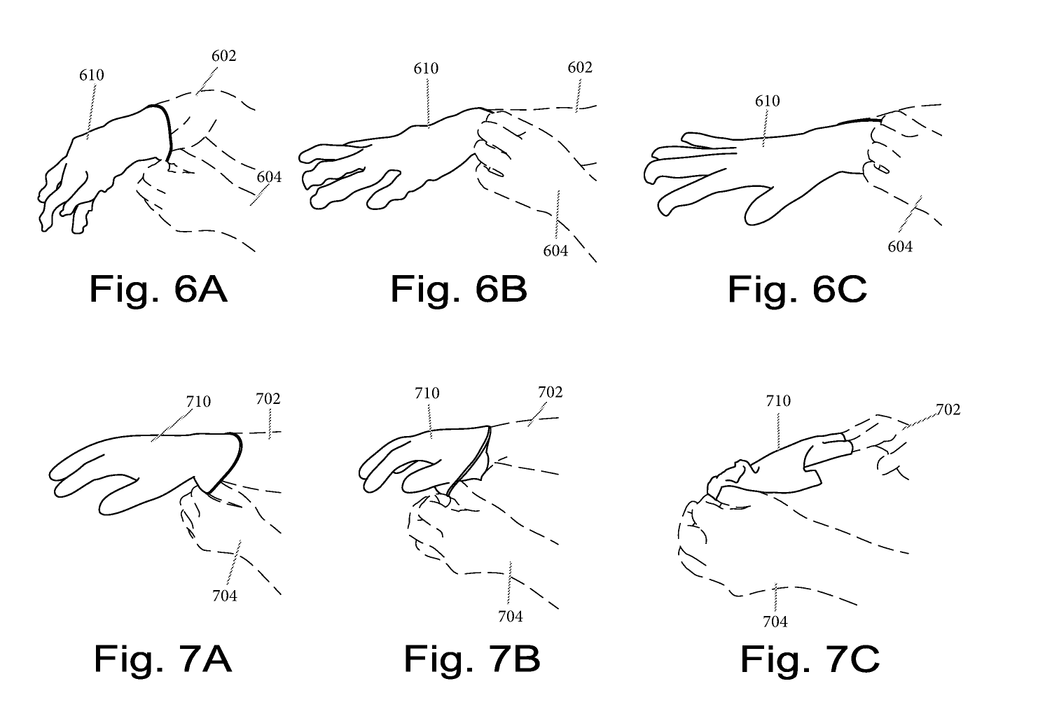

FIGS. 6A through 6C are illustrations showing the process of donning a conventional medical glove. FIG. 6A shows how the wearer uses his/her left hand 604 to don the glove 610 on the right hand 602. The wearer inserts his/her thumb on hand 604 into the glove 610 to pull it onto the hand 602. FIG. 6B shows how the glove is halfway donned on hand 602 as hand 604 pulls on the glove and FIG. 6C shows the glove 610 is completely donned on hand 602 as hand 604 has completed pulling on the glove 610. Note that this process requires that the wearer insert his/her thumb on hand 604 into the glove 610 to pull it onto the hand 602.

FIGS. 7A through 7C are illustrations showing the process of removing a conventional medical glove. FIG. 7A shows how the wearer uses his/her left hand 704 to remove the glove 710 from the right hand 702. The wearer inserts his/her thumb on hand 704 into the glove 710 to pull it off the hand 702. FIG. 7B shows how the glove is halfway removed from hand 702 as hand 704 removes the glove and FIG. 7C shows the glove 710 is completely removed from hand 702 as hand 704 has completed removed the glove 710. Note that this process requires that the wearer insert his/her thumb on hand 704 into the glove 710 to remove the glove, which could result in contaminating the bare hand 702 with any material that may be present.

FIG. 8A is an illustration of the beginning of the process of removing the claimed protective gloves, according to an example embodiment. FIG. 8A shows how the wearer may use a first hand 802 to grab the bulging portion 812 on the glove of the second hand 804, so as to pinch the bulging portion between his/her fingers, and allow significant purchase on the glove on the second hand 804. FIG. 8A also shows how the wearer may use his/her second hand 804 to grab the bulging portion 814 on the first hand 802, so as to pinch the bulging portion between his/her fingers, and allow significant purchase on the glove on the first hand 802. In this manner, the wearer can start the process of removing the gloves on his/her own, without the need for a companion or helper, in such a way that he does not contaminate himself or others. The glove removal process progresses with reference to FIGS. 8B through 8C below.

FIG. 8B is an illustration of the middle of the process of removing the claimed protective gloves, according to an example embodiment. FIG. 8B shows how the wearer, using first hand 802, continues to pull the bulging portion 812 on the glove of the second hand 804, so as to start the removal of the glove from the second hand 804. FIG. 8B also shows how the wearer, using his/her second hand 804, continues to pull the bulging portion 814 on glove of the first hand 802, so as to start the removal of the glove on the first hand 802. The glove removal process progresses with reference to FIG. 8C below.

FIG. 8C is an illustration further in the process of removing the claimed protective gloves, according to an example embodiment. FIG. 8C shows how the wearer, using first hand 802, is pulling off the glove of the second hand 804, without allowing the outer surface of the glove on the second hand 804 to touch his/her bare skin (or the bare skin of another person). FIG. 8C also shows how the wearer, using his/her second hand 804, is pulling off the glove of the first hand 802, without allowing the outer surface of the glove on the first hand 802 to touch his/her bare skin (or the bare skin of another person). Note that the two gloves are being pulled off at the same time (i.e., simultaneously) by the wearer.

The claimed protective gloves may be disposable gloves and may be used during medical examinations and procedures that help prevent cross-contamination between caregivers and patients. The claimed protective gloves may be made of different polymers including latex, nitrile rubber, vinyl and neoprene, as well as high grade isoprene, and artificial polyisoprene. The claimed protective gloves may be provided un-powdered, or powdered with cornstarch to lubricate the gloves, making them easier to put on the hands. Alternatively, lycopodium powder and talc may be used. To make them easier to don without the use of powder, the claimed protective gloves can be treated with chlorine. Chlorination affects some of the beneficial properties of latex, but also eliminates soluble proteins that cause allergic reactions. The claimed protective gloves may be either sterile or non-sterile.

The manufacturing of the claimed protective gloves may be executed as follows. The manufacturing equipment may run ceramic, hand-shaped formers through water and bleach to clean them and remove any residue from the previous manufacturing run. The formers may then dry to remove all the water used to clean the formers. Then, the formers are dipped in a mixture of calcium carbonate and calcium nitrate, which helps the synthetic materials coagulate around the formers. Afterward, the formers are once again dried. The manufacturing equipment may then dip the formers in tanks full of nitrile butadiene rubber or polyvinyl chloride. The following step involves heating the materials at a high temperature to form the gloves as they dry. For easier donning, nitrile gloves undergo one of two processes: chlorination or polymer coating. Chlorination involves exposing the gloves to chlorine—as an acid mixture or gas—to make the material harder and slicker. Polymer coating lubricates the glove surface by adding a layer of polymer. Finally, in what is known as the stripping phase, the gloves are removed from the formers.

The last steps of the manufacturing cycle may include testing the gloves. The quality control process may include a pinhole leak test. This test determines whether a glove has enough pinholes to lead to a noticeable leak. After filling the gloves with 1 liter of water, the gloves are hung upside down for two minutes to determine if the gloves can hold the water. If a noticeable leak is detected, the gloves do not pass testing.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

What is claimed is:

1. A pair of protective gloves, each glove comprising:
(a) a hollow-formed, seamless and flexible body element composed of a polymer, the flexible body element including a closed distal end for containing the wearer's hand, an open proximal end, and a cuff portion at said proximal end, wherein the flexible body element is configured to conform tightly to the wearer's hand;
(b) a bulging portion of the flexible body element located on the cuff portion, said bulging portion extending around the cuff to form a ring around the cuff, and said bulging portion projecting outwards from said body element so as not to conform to the wearer's hand;
(c) wherein said bulging portion comprising loose material that may be pinched between the wearer's fingers, so as to allow significant purchase on the glove; and
(d) wherein said bulging portion being positioned so that when said glove is properly positioned on the wearer's hand, said bulging portion may be pulled in a substantially distal direction, thereby removing the glove from the wearer's hand without the exterior surface of the glove touching the wearer's hand.

2. The pair of protective gloves of claim 1, wherein the cuff portion is located at last one inch from the proximal end.

3. The pair of protective gloves of claim 2, wherein the bulging portion comprises irregularly shaped loose material.

4. The pair of protective gloves of claim 3, wherein the flexible body element is further composed of latex.

5. The pair of protective gloves of claim 3, wherein the flexible body element is further composed of nitrile rubber.

6. The pair of protective gloves of claim 2, wherein the bulging portion comprises a set of elliptical shaped protrusions that extend around the cuff.

7. The pair of protective gloves of claim 6, wherein the flexible body element is further composed of latex.

8. The pair of protective gloves of claim 7, wherein the flexible body element is further composed of nitrile rubber.

9. The pair of protective gloves of claim 2, further comprising a layer of cornstarch applied to the gloves.

10. The pair of protective gloves of claim 2, wherein the polymer has been chlorinated.

11. A pair of protective gloves, each glove comprising:
(a) a hollow-formed, seamless and flexible body element composed of a polymer, the flexible body element including a closed distal end for containing the wearer's hand, an open proximal end, and a cuff portion about an inch from said proximal end, wherein the flexible body element is configured to conform tightly to the wearer's hand;
(b) a protruding portion of the flexible body element located on the cuff portion, said protruding portion projecting outwards from said body element;
(c) wherein said bulging portion comprising loose material that may be pinched between the wearer's fingers, so as to allow significant purchase on the glove; and
(d) wherein said bulging portion being positioned so that when said glove is properly positioned on the wearer's hand, said bulging portion may be pulled in a substantially distal direction, thereby removing the glove from the wearer's hand without the exterior surface of the glove touching the wearer's hand.

12. The pair of protective gloves of claim 11, wherein the cuff portion is located at last one inch from the proximal end.

13. The pair of protective gloves of claim 12, wherein the bulging portion comprises irregularly shaped loose material.

14. The pair of protective gloves of claim 13, wherein the flexible body element is further composed of latex.

15. The pair of protective gloves of claim 13, wherein the flexible body element is further composed of nitrile rubber.

16. The pair of protective gloves of claim 12, wherein the bulging portion comprises a set of elliptical shaped protrusions that extend around the cuff.

17. The pair of protective gloves of claim 16, wherein the flexible body element is further composed of latex.

18. The pair of protective gloves of claim 17, wherein the flexible body element is further composed of nitrile rubber.

19. The pair of protective gloves of claim 12, further comprising a layer of cornstarch applied to the gloves.

20. The pair of protective gloves of claim 12, wherein the polymer has been chlorinated.
